SEQUENCE LISTING (7 sequences)

SEQ ID NO: 1 - protein, 371 aa, Streptococcus pyogenes
DQNFARNEKEAKDSAITFIQKSAAIKAGARSAEDIKLDKVNLGGELSGSNMYVYNISTGGFVIVSGDKRSPEILGYSTSGSFDANGKENIASFMESYVEQIKENKKLDTTYAGTAEIKQPVVKSLLDSKGIHYNQGNPYNLLTPVIEKVKPGEQSFVGQHAATGCVATATAQIMKYHNYPNKGLKDYTYTLSSNNPYFNHPKNLFAAISTRQYNWNNILPTYSGRESNVQKMAISELMADVGISVDMDYGPSSGSAGSSRVQRALKENFGYNQSVHQINRSDFSKQDWEAQIDKELSQNQPVYYQGVGKVGGHAFVIDGADGRNFYHVNWGWGGVSDGFFRLDALNPSALGTGGGAGGFNGYQSAVVGIKP

SEQ ID NO: 2 - protein, 310 aa, Streptococcus pyogenes
DSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 3 - protein, 11 aa, Homo sapiens
CPPCPAPELLG

SEQ ID NO: 4 - protein, 20 aa, Homo sapiens
KTHTCPPCPAPELLGGPSVF

SEQ ID NO: 5 - protein, 373 aa, Streptococcus pyogenes
MADQNFARNEKEAKDSAITFIQKSAAIKAGARSAEDIKLDKVNLGGELSGSNMYVYNISTGGFVIVSGDKRSPEILGYSTSGSFDANGKENIASFMESYVEQIKENKKLDTTYAGTAEIKQPVVKSLLDSKGIHYNQGNPYNLLTPVIEKVKPGEQSFVGQHAATGCVATATAQIMKYHNYPNKGLKDYTYTLSSNNPYFNHPKNLFAAISTRQYNWNNILPTYSGRESNVQKMAISELMADVGISVDMDYGPSSGSAGSSRVQRALKENFGYNQSVHQINRSDFSKQDWEAQIDKELSQNQPVYYQGVGKVGGHAFVIDGADGRNFYHVNWGWGGVSDGFFRLDALNPSALGTGGGAGGFNGYQSAVVGIKP

SEQ ID NO: 6 - protein, 339 aa, Streptococcus pyogenes
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 7 - protein, 451 aa, Homo sapiens
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2016/0231329 A1
(43) Pub. Date: Aug. 11, 2016

(54) A METHOD FOR ANALYSING A SAMPLE IMMUNOGLOBULIN MOLECULES

(71) Applicant: GENOVIS AB, Lund (SE)

(72) Inventors: Fredrik OLSSON, Kavlinge (SE); Maria NORDGREN, Hassleholm (SE); Malin MEJARE, Dalby (SE); Sarah FREDRIKSSON, Dalby (SE)

(21) Appl. No.: 15/023,042

(22) PCT Filed: Sep. 18, 2014
(86) PCT No.: PCT/EP2014/069920

§ 371 Date: Mar. 18, 2016

(30) Foreign Application Priority Data
Sep. 20, 2013 (GB) 1316744.0

(51) Int. Cl. G01N 33/68; C12N 9/64; C07K 16/00

(52) U.S. Cl. CPC G01N 33/6857; C07K 16/00; C12N 9/6472; C07K 2317/53; C12Y 304/2201

(57) ABSTRACT

The inventions provides methods for analysing a sample of immunoglobulins, related peptides, and kits for carrying out such methods.

### FIELD OF THE INVENTION

The present invention relates to methods for analysing a sample of immunoglobulins, to related peptides, and to kits for carrying out such methods.

### BACKGROUND OF THE INVENTION

The characterisation of antibodies, such as structural characterisation and physiochemical analysis, is required by developers and producers of antibody based therapeutics. Mass spectrometry (MS) is one of the key analytical tools for characterizing therapeutic monoclonal antibodies (MAbs). Mass spectrometry in conjugation with HPLC is commonly used for studying the primary structure as well as post translation modifications (PTMs) and glycan structures of these large biomolecules. The large size of MAbs (150 kD) together with post translational modifications (PTMs) makes the analytical characterization of these biological therapeutics especially challenging.

For example, antibody-drug conjugates (ADCs) are an important class of therapeutic agent, which harness the selectivity of monoclonal antibodies (mAbs) to achieve targeted delivery of therapeutic agents. Critical to the clinical efficacy of an ADC are the target site-specificity and binding properties of the antibody, the in vitro and in vivo stability of the linker and the therapeutic agent, the potency of the therapeutic agent, and both the distribution and average number of therapeutic agents on the antibody. It is therefore important to understand the physiochemical properties of ADCs and develop analytical and bioanalytical techniques to assess and monitor ADCs during manufacture and subsequent storage.

### SUMMARY OF THE INVENTION

Streptococcal erythrogenic toxin B (SpeB) is a cystein protease from Streptococcus pyogenes, shown to cleave IgG in the hinge region into two stable monomeric Fab fragments and one Fc fragment. In particular, SpeB has been reported to cleave human IgG between glycine residues 236 and 237. A second cystein protease from Streptococcus pyogenes, Immunoglobulin G-degrading enzyme of S. pyogenes (IdeS) has been reported to have an identical cleavage site for IgG as SpeB.

The inventors have carefully examined SpeB and IdeS activity on IgG and have made the surprising discovery that SpeB in fact cleaves IgG at a different site in the hinge region than IdeS. Developing this surprising discovery, the inventors realised that tandem cleavage of immunoglobulin with SpeB and IdeS allows the investigation of the hinge region of immunoglobulin molecules in more detail than was previously thought possible. This is important because the hinge region is a key site for post-translational modification and for conjugation of therapeutic agents in antibody-drug conjugates. Accordingly, the invention provides:

A method for analysing a sample of immunoglobulin molecules, comprising contacting the sample with a first polypeptide and a second polypeptide and analysing the resulting mixture, wherein the first polypeptide and the second polypeptide are cysteine protease enzymes which each cleave a different target site in the hinge region of human IgG;

A peptide consisting of the amino acid sequence CPPCPAPELLG (SEQ ID NO: 3), or a variant of said sequence comprising one or two conservative modifications; and

A kit for use in a method of analysing a sample of immunoglobulin molecules, the kit comprising a first polypeptide and a second polypeptide as defined above.

### BRIEF DESCRIPTION OF THE FIGURES

FIGS. 1A and 1B show results from SDS-PAGE following cleavage of the human monoclonal IgG1 antibody herceptin with IdeS (FabRICATOR) alone, SpeB (Fpn-1) and IdeS together, or SpeB alone.

FIG. 2 shows a schematic overview of the different cleavage sites for SpeB (Fpn-1) and IdeS for human monoclonal IgG1 (Herceptin), as determined by mass spectrometry (LC/MS).

FIG. 3 shows a schematic overview of cleavage of human IgG by SpeB (Fpn-1) giving rise to Fab and Fc fragments. The novel SpeB cleavage site at the hinge region is also indicated.

FIGS. 4A and 4B show a RP chromatogram with overlay of the hinge regions of peptides from avastin, herceptin and adcetris antibodies. In FIG. 4B, a control synthetic hinge peptide has been added.

### BRIEF DESCRIPTION OF THE SEQUENCES

SEQ ID NO:1 is an amino acid sequence of S. pyogenes SpeB.

SEQ ID NO:2 is an amino acid sequence encoding IdeS isolated from S. pyogenes AP1.

SEQ ID NO:3 is an amino acid sequence of the peptide that results from cleavage of an exemplary IgG molecule (herceptin) with SpeB and IdeS.

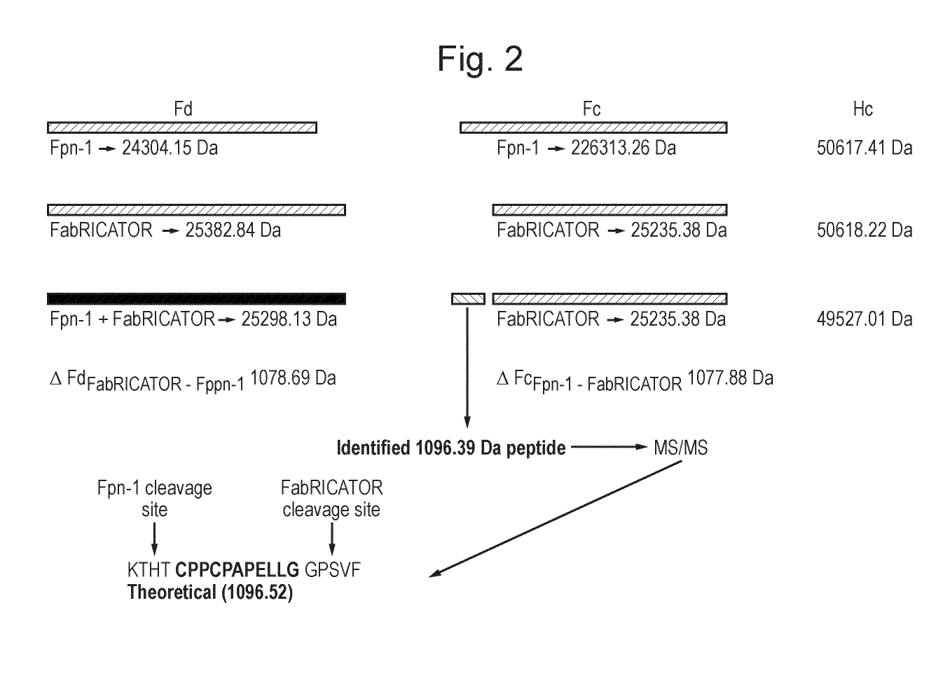

SEQ ID NO: 4 is an amino acid sequence of part of the hinge region of an exemplary IgG molecule (herceptin). This sequence comprises the SpeB (Fpn-1) and IdeS (FabRICATOR) cleavage sites (as shown in FIG. 2).

SEQ ID NO:5 is an amino acid sequence of S. pyogenes SpeB, including a Met Ala N-terminal to the SpeB sequence.

SEQ ID NO:6 is an amino acid sequence encoding IdeS isolated from S. pyogenes AP1, including a putative signal sequence.

SEQ ID NO:7 is the amino acid sequence of the anti-HER2 heavy chain 1 of an exemplary IgG molecule (herceptin), which includes the hinge region recognised by SpeB and IdeS.

### DETAILED DESCRIPTION OF THE INVENTION

It is to be understood that different applications of the disclosed methods and products may be tailored to the specific needs in the art. It is also to be understood that the terminology used herein is for the purpose of describing particular embodiments of the invention only, and is not intended to be limiting. In addition as used in this specification and the appended claims, the singular forms “a”, “an”, and “the” include plural referents unless the content clearly dictates otherwise. Thus, for example, reference to “an immunoglobulin” includes two or more such immunoglobulins, and the like. All publications, patents and patent applications cited herein, whether supra or infra, are hereby incorporated by reference in their entirety.

The invention provides a method for analysing a sample of immunoglobulin molecules, comprising contacting the sample with a first polypeptide and a second polypeptide. The sample typically contains at least one IgG molecule, and the method is typically carried out ex vivo, preferably in vitro.

The first polypeptide and the second polypeptide are enzymes, specifically cysteine protease enzymes, which cleave IgG, preferably human IgG, in the hinge region of the heavy chain. The first and second polypeptides target different cleavage sites in the hinge region of the heavy chain of IgG. Accordingly, contacting a sample of immunoglobulin molecules with the first polypeptide and the second polypeptide results in a mixture of molecules of various sizes, which may be analysed to provide information about the original sample. The mixture particularly includes a short peptide from the hinge region of the heavy chain of IgG which lies between the cleavage site of the first polypeptide and the cleavage site of the second polypeptide. The method of the invention typically determines the presence, absence and/or amount of this short peptide. The method of the invention may further analyse the short peptide, for example, to determine the presence or absence of post-translational modifications and/or conjugated moieties such as therapeutic agents.

The first polypeptide is typically a SpeB polypeptide, preferably a SpeB polypeptide from S. pyogenes. The first polypeptide is preferably not papain. The first polypeptide may be a SpeB polypeptide from another organism, such as another Streptococcus bacterium, for example Streptococcus thermophilius. The first polypeptide preferably comprises or consists of the amino acid sequence set forth in SEQ ID NOs:1 or 5.

The first polypeptide cleaves the hinge region of IgG between positions 238 and 239 according to the Kabat numbering system (positions 225 and 226 according to EU numbering system). By way of example, SpeB cleaves between amino acid numbers 229 and 230 of SEQ ID NO:7 (the anti-HER2 heavy chain 1 of herceptin). A SpeB polypeptide may be obtained by any suitable means. For example, it may be isolated from any suitable organism that expresses it, such as the S. pyogenes bacterium. SpeB polypeptides are commercially available.

The second polypeptide is typically an IdeS polypeptide, preferably an IdeS polypeptide from S. pyogenes. The second polypeptide may be an IdeS polypeptide from another organism, such as another Streptococcus bacterium. The Streptococcus is preferably a group A Streptococcus, a group C Streptococcus or a group G Streptococcus. In particular, the second polypeptide may be an IdeS polypeptide from a group C Streptococcus such as S. equii or S. zooepidemicus. Alternatively, the second polypeptide may be from Pseudomonas putida. The second polypeptide preferably comprises or consists of the amino acid sequence set forth in SEQ ID NOs: 2 or 6.

The second polypeptide cleaves the hinge region of IgG between positions 249 and 250 according to the Kabat numbering system (positions 236 and 237 according to EU numbering system). By way of example, IdeS cleaves between amino acid numbers 240 and 241 of SEQ ID NO:7 (the anti-HER2 heavy chain 1 of herceptin). An IdeS polypeptide may be obtained by any suitable means. For example, it may be isolated from any suitable organism that expresses it, such as the S. pyogenes bacterium. IdeS polypeptides are commercially available.

A sequence taken from the hinge region of an exemplary IgG molecule (herceptin) is shown below to illustrate the different cleavage sites of the first and the second polypeptide. The first polypeptide cleaves between the two italic underlined residues. The second polypeptide cleaves between the two bold underlined residues.

(SEQ ID NO: 4)

KTHTCPPCPAPELLGGPSVF

Cleavage of an IgG molecule comprising this sequence with the first polypeptide and the second polypeptide results in a novel short peptide. Said peptide corresponds to residues 239 to 249 of the hinge region according to the Kabat numbering system (residues 226 to 236 according to EU numbering). Said peptide will typically have a molecular weight of approximately 1096 Da (nearest Da) and may typically consist of the sequence CPPCPAPELLG (SEQ ID NO: 3). As will be appreciated, the molecular weight of the short peptide will be altered by the presence of another moiety (such as a therapeutic agent) conjugated to any one of residues 239 to 249 (Kabat numbering), or by post-translation modification (such as glyosylation) of any one of those residues. The cleavage sites of the first and second polypeptides are further illustrated in FIG. 2.

For the purposes of the method of the invention, the first polypeptide and/or the second polypeptide may be replaced with a variant or fragment of each thereof, provided said variant or fragment retains the functional characteristics of the original polypeptide. Specifically, the variant or fragment of the first polypeptide must retain the IgG cysteine protease activity and cleave IgG at the same site as the first polypeptide. The variant or fragment of the second polypeptide must retain the IgG cysteine protease activity and cleave IgG at the same site as the second polypeptide.

The cysteine protease activity of any polypeptide may be determined by means of a suitable assay. For example, a test polypeptide may be incubate with IgG at a suitable temperature, such as 37° C. The starting materials and reaction products may then be analysed by SDS-PAGE to determine whether the desired IgG cleavage product is present. The cleavage product may be subjected to N-terminal sequencing to verify that cleavage has occurred in the hinge region of IgG. The cysteine protease activity of the polypeptide can be further characterised by inhibition studies. Preferably, the activity is inhibited by the peptide derivative Z-LVG-CHN₂ and/or by iodoacetic acid both of which are protease inhibitors. However, for the second polypeptide (or a variant or fragment thereof) the activity is generally not inhibited by E64.

Retention of a specific cleavage site of a polypeptide may also be determined by any suitable means. For example it may be determined by comparing the fragments which result from cleavage of IgG with the polypeptide, to the fragments which result from cleavage of IgG with a polypeptide for which the cleavage site has previously been confirmed. For example, a variant or fragment of the first polypeptide should produce the same fragments as the polypeptide of SEQ ID NOs: 1 or 5. A variant or fragment of the second polypeptide should produce the same fragments as the polypeptide of SEQ ID NOs: 2 or 6.

Variants of the first polypeptide may include polypeptides which have at least 80%, at least, 85%, preferably at least 90%, at least 95%, at least 98% or at least 99%, identity to SEQ ID NOs:1 or 5. The identity of variants of SEQ ID NOs: 1 or 5 can be measured over a region of at least 50, at least 100, at least 200, at least 300 or more contiguous amino acids of the sequence shown in SEQ ID NOs: 1 or 5, or more preferably over the full length of SEQ ID NOs: 1 or 5.

Variants of the second polypeptide may include polypeptides which have at least 80%, at least, 85%, preferably at least 90%, at least 95%, at least 98% or at least 99% identity to SEQ ID NOs:2 or 6. The identity of variants of SEQ ID NOs: 2 or 6 can be measured over a region of at least 50, at least 100, at least 200, at least 300 or more contiguous amino acids of the sequence shown in SEQ ID NOs: 2 or 6, or more preferably over the full length of SEQ ID NOs: 2 or 6.

Amino acid identity may be calculated using any suitable algorithm. For example the PILEUP and BLAST algorithms can be used to calculate identity or line up sequences (such as identifying equivalent or corresponding sequences (typically on their default settings), for example as described in Altschul S. F. (1993) J Mol Evol 36:290-300; Altschul, S, F et at (1990) J Mol Biol 215:403-10. Software for performing BLAST analyses is publicly available through the National Center for Biotechnology Information (http://www.ncbi.nlm.nih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89: 10915-10919) alignments (B) of 50, expectation (E) of 10, M=5, N=4, and a comparison of both strands.

The BLAST algorithm performs a statistical analysis of the similarity between two sequences; see e.g., Karlin and Altschul (1993) Proc. Natl. Acad. Sci. USA 90: 5873-5787. One measure of similarity provided by the BLAST algorithm is the smallest sum probability (P(N)), which provides an indication of the probability by which a match between two polynucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001. Alternatively, the UWGCG Package provides the BESTFIT program which can be used to calculate identity (for example used on its default settings) (Devereux et at (1984) Nucleic Acids Research 12, 387-395).

Variants may include allelic variants and the substitution, deletion or insertion of single amino acids or groups of amino acids within the protein sequence. Variant sequences may differ by at least 1, 2, 5, 10, 20, 30, 50 or more mutations (which may be substitutions, deletions or insertions of amino acids) when compared to an original sequence. For example, from 1 to 50, 2 to 30, 3 to 20 or 5 to 10 amino acid substitutions, deletions or insertions may be made. Substitution variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala | aliphatic, hydrophobic, neutral | Met | hydrophobic, neutral |
| Cys | polar, hydrophobic, neutral | Asn | polar, hydrophilic, neutral |
| Asp | polar, hydrophilic, charged (−) | Pro | hydrophobic, neutral |
| Glu | polar, hydrophilic, charged (−) | Gln | polar, hydrophilic, neutral |
| Phe | aromatic, hydrophobic, neutral | Arg | polar, hydrophilic, charged (+) |
| Gly | aliphatic, neutral | Ser | polar, hydrophilic, neutral |
| His | aromatic, polar, hydrophilic, | Thr | polar, hydrophilic, neutral |
| | charged (+) | | |
| Ile | aliphatic, hydrophobic, neutral | Val | aliphatic, hydrophobic, neutral |
| Lys | polar, hydrophilic, charged(+) | Trp | aromatic, hydrophobic, neutral |
| Leu | aliphatic, hydrophobic, neutral | Tyr | aromatic, polar, hydrophobic |

Fragments of the first polypeptide typically consist of no more than 100, 150, 200, 250, 300 or 350 contiguous amino acids of SEQ ID NO:s 1 or 5. Fragments of the second polypeptide typically consist of no more than 100, 150, 200, 250, 300 or 350 contiguous amino acids of SEQ ID NOs:2 or 6.

The amino acid sequence of any polypeptide, variant or fragment as described herein may be modified to include non-naturally occurring amino acids and/or to increase the stability of the compound. When the polypeptides are produced by synthetic means, such amino acids may be introduced during production. The polypeptides may also be modified following either synthetic or recombinant production. The polypeptides, variants or fragments described herein may be produced using D-amino acids. In such cases the amino acids will be linked in reverse sequence in the C to N orientation. This is conventional in the art for producing such polypeptides. A number of side chain modifications are known in the art and may be made to the side chains of the polypeptides, variants or fragments, subject to their retaining any further required activity or characteristic as may be specified herein. It will also be understood that the polypeptides, variants or fragments may be chemically modified, e.g. post-translationally modified. For example, they may be glycosylated, phosphorylated or comprise modified amino acid residues.

The immunoglobulin containing sample used in the method of the invention may include immunoglobulin molecules such as IgM, IgA, IgD, and/or IgW, provided it includes at least one IgG molecule. Said IgG may be from any species, for example, human, monkey, rabbit, sheep or mouse, but is preferably human. Said IgG may be humanized or chimeric. The IgG may be Mouse IgG2a or IgG3. Preferably, the IgG is human IgG1, IgG2, IgG3 or IgG4.

Any suitable sample containing immunoglobulin molecules may be used in the method of the invention. The sample is typically a fluid. For example, the sample may be a blood, serum or saliva sample. Alternatively the sample may be taken from a batch of synthetically produced immunoglobulins, or may be formulated for administration to a patient with a pharmaceutical carrier or diluent. The sample may thus comprise any therapeutic monoclonal antibody or antibody-drug conjugate. For example, the sample may comprise molecules of avastin, herceptin or adcetris.

The sample preferably comprises at least one human IgG molecule conjugated to a therapeutic agent. Preferably, the human IgG molecule is conjugated to the therapeutic agent via the thiol group of a cysteine residue. Preferably, the cysteine residue is in the hinge region of the human IgG molecule, most preferably between residues 239 and 249 (Kabat numbering system). Preferably the therapeutic agent is a cytotoxin. Suitable toxins include avristatin, calicheamicins, CC-1065, doxorubicin, maytonsinoid, methotrexate and vinca alkaloids.

The method of the invention may comprise the following steps:

(a) contacting the sample with the first polypeptide;

(b) isolating Fc fragments from the resulting mixture;

(c) contacting the isolated Fc fragments with the second polypeptide; and

(d) analysing the resulting mixture.

Step (a) may be performed under any conditions that permit the cleavage of immunoglobulin molecules in the sample by the first polypeptide. Suitable conditions are described in the Examples. Typically, any standard buffer is used at a pH of 6.5 to 8.0. Standard buffers include phosphate buffer saline (PBS), tris, ammonium bicarbonate, MES, HEPEs and sodium acetate. Typically, the sample is incubated with the first polypeptide for at least 20 minutes, at least 30 minutes, at least 40 minutes, at least 50 minutes, preferably at least 60 minutes. Incubation preferably takes place at room temperature, more preferably at approximately 20° C., 25° C., 30° C., 35° C., 40° C. or 45° C., and most preferably at approximately 37° C. Typically, the enzyme:antibody ratio is approximately 1:50 (w:v). Typically, a reducing agent, such as iodocetamide, DTT or TCEP is used.

The separation of Fc fragments in step (b) may be performed using any suitable method. For example, Fc fragments may be separated from the resulting mixture by affinity separation, size-exclusion chromatography (SEC), ion-exchange chromatography, gel filtration or dialysis. Typically, the mixture may be contacted with a suitable Fc binding agent. The mixture resulting from step (a) may be applied onto a human IgG Fc-binding resin and components other than Fc fragments, which do not bind to the resin (such as, for example, Fab fragments, the reducing agent and SpeB), can be eluted off. Fc-binding agents such as human IgG Fc-binding resin are commercially available.

Step (c) may be performed under any conditions that permit the cleavage of Fc fragments by the second polypeptide. Suitable conditions are described in the Examples. Typically, any standard buffer is used, as described above. Typically, the sample is incubated with the IdeS polypeptide for at least 20 minutes, at least 30 minutes, at least 40 minutes, at least 50 minutes, preferably at least 60 minutes. Incubation preferably takes place at room temperature, more preferably at approximately 20° C., 25° C., 30° C., 35° C., 40° C. or 45° C., and most preferably at approximately 37° C. Typically, the enzyme:antibody ratio is approximately 1:50 (w:v). Typically, a reducing agent is not used.

Step (c) may optionally further comprise removing Fc fragments according to any suitable method, for example by applying the mixture resulting from step (c) to a human IgG Fc-binding resin. Fc fragments will be retained and other molecules (including, for example, the second polypeptide and the 1096 Da peptide) will be eluted and may then be isolated.

The method may alternatively comprise the following steps:

(a) contacting the sample with the second polypeptide;

(b) isolating Fab fragments from the resulting mixture;

(c) contacting the isolated Fab fragments with the first polypeptide; and

(d) analysing the resulting mixture.

Step (a) may be performed under any conditions that permit the cleavage of immunoglobulin molecules in the sample by the second polypeptide. Suitable conditions are as described above.

The separation of Fab fragments in step (b) may be performed using any suitable method. For example, Fab fragments may be separated from the resulting mixture using the methods described above for separating Fc fragments. Typically, any suitable Fab binding agent may be used.

Step (c) may be performed under any conditions that permit the cleavage of a Fab fragment by the first polypeptide. Suitable conditions are as described above for the cleavage of whole immunoglobulins by the first polypeptide.

Step (c) may optionally further comprise removing Fab fragments according to any suitable method, for example by applying the mixture resulting from step (c) to a suitable Fab binding agent. Fab fragments will be retained and other molecules (including, for example, the first polypeptide and the 1096 Da peptide) will be eluted and may then be isolated.

The method may alternatively comprise the following steps:

(a) contacting the sample with both the first and the second polypeptide; and

(b) analysing the resulting mixture.

Step (a) is performed under conditions that permit the cleavage of immunoglobulins in the sample by the first and second polypeptides. Suitable conditions are as described above.

Irrespective of the preceding steps described above, any suitable method may be used in analysing the resulting final mixture. Typically, analysing the resulting mixture comprises determining the molecular weight of at least one molecule, preferably using HPLC and/or mass spectrometry.

The analysis of the resulting mixture may be carried out to determine:

(a) the proportion of immunoglobulin molecules in the sample to which a therapeutic agent is conjugated; and/or

(b) the ratio of therapeutic agent:immunoglobulin molecule; and/or

(c) the presence or absence of post translational modifications.

Determining the proportion of immunoglobulin molecules in the sample to which a therapeutic agent is conjugated and/or the ratio of therapeutic agent:immunoglobulin molecule may help determine the amount of therapeutic agent that can be delivered to the site of interest, and may directly affect both safety and efficacy of the sample. Typical methods include UV/VIS, UV/MALDI and/or UV/DAR spectroscopy and hydrophobic interaction chromatography (HIC) analysis.

The resulting mixture may typically be analysed for the presence, absence, and/or amount of a peptide with a molecular weight of approximately 1096 Da. As explained above and shown in the Examples, cleavage of human IgG in accordance with the method of the invention will typically result in such a peptide.

In a specific embodiment (referred to as “off-line” characterisation) the mixture is analysed using HPLC only. This embodiment is typically used when an immunoglobulin or antibody-drug conjugate has previously been fully characterised (e.g. by both HPLC and mass spectrometry) because each peak in the HPLC chromatogram will already be known and can be defined with a high precision and accuracy. In such a case, provided the peaks appear at the predicted positions, a sample can be considered to be consistent with the previously characterised sample. If not, additional analysis of the sample may be required to determine what is different. For example, mass spectrometry may be used to further characterise the sample. This embodiment may be particularly useful where the same antibody is routinely mass-produced, and periodically a sample is tested for quality control purposes.

Typical methods for determining the presence or absence of post translational modifications, such as pyroglutamic acid formation, oxidation, deamidation, isomerization, glycation, disulfide shuffling, peptide bond cleavage and cross-linkage include capillary electrophoresis (CE), capillary liquid chromatography (CLC), UV absorbance and laser-induced fluorescence (LIF).

The invention also provides an isolated peptide having the sequence of SEQ ID NO:3, or a variant of said sequence comprising one or two conservative modifications, preferably only within positions 2 to 10 of the sequence. The peptide may be produced by treatment of an immunoglobulin containing sample with the first and second polypeptides of the invention. The invention further provides said peptide conjugated to any therapeutic agent, as defined above.

The invention also provides kits comprising the first and second polypeptide of the invention. Said kits may be used in the method of the invention. The following Examples illustrate the invention:

### Example 1

SpeB activity on human IgG has been examined using SDS-PAGE and Mass spectrometry. It has been found that the cleavage site of SpeB is unexpectedly different from that previously reported.

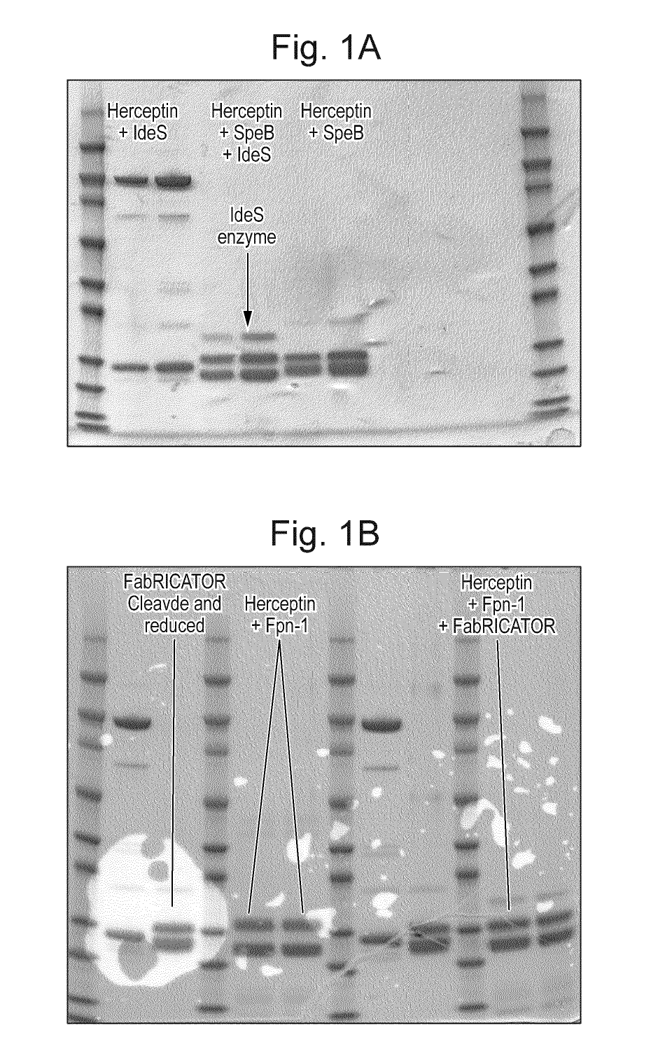

Human monoclonal IgG (Herceptin) was incubated with IdeS, SpeB or a combination of both enzymes. SDS-PAGE analysis (FIG. 1) indicates that the cleavage site is not the same for both enzyme, contrary to previous reports. A mass shift observed on SDS-PAGE on the combination of IdeS and SpeB when compared with IdeS and SpeB alone indicates that the cleavage site is different. This also shows that IdeS (added subsequently to the reaction) can cleave the fragment generated by SpeB.

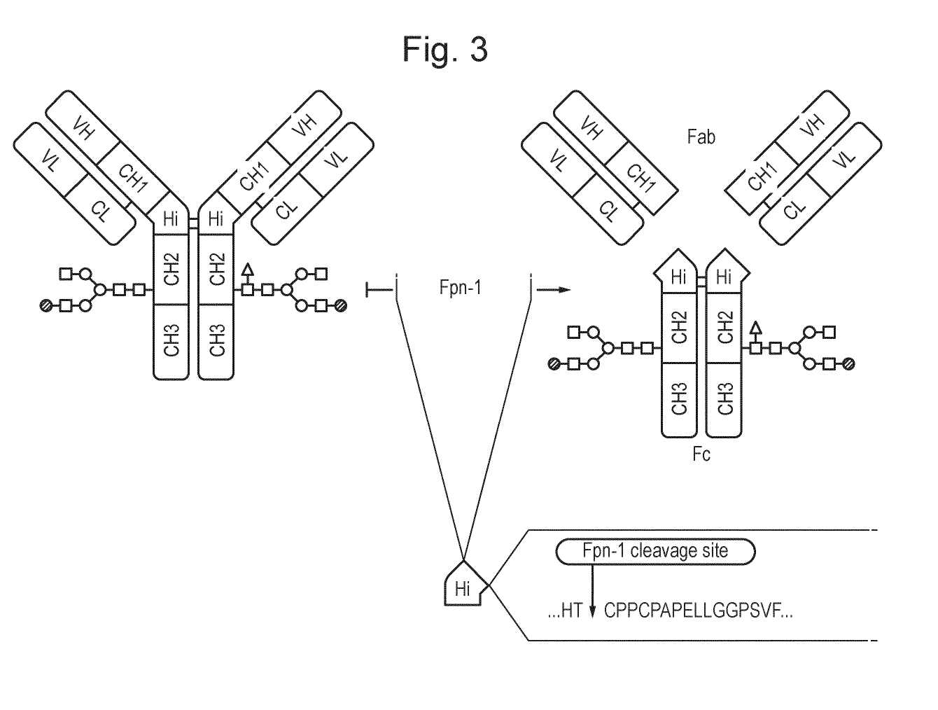

To further investigate the cleavage site of IdeS and SpeB liquid chromatography in combination with mass spectrometry (LC/MS) was employed to verify the above-mentioned results from SDS-PAGE and to reveal the detail of the cleavage site. The results of the LC/MS analysis are summarised in FIG. 2, and confirm different cleavage sites for SpeB and IdeS. A schematic overview of SpeB is shown in FIG. 3.

### Example 2

The hinge region peptide CPPCPAPELLG (as set forth in SEQ ID NO:3) was prepared from antibody samples.

Fc fragments of antibody samples (avastin, herceptin and adcetris) were initially isolated using His-tagged recombinant SpeB enzyme (also referred to as Fpn-1). This cleavage reaction was performed in a standard buffer at pH 6.5 to 8.0, using an enzyme:antibody ratio 1:50 (w:w) and the reducing agent DTT or TCEP at 1-5 mM for 1 h at 37° C. After cleavage was completed, material from the entire reaction was applied onto Capture Select human IgG Fc resin and eluted free from Fab fragments, reducing agent and IdeS enzyme.

The eluted Fc was cleaved with His-tagged recombinant IdeS enzyme (also referred to as FabRICATOR) in a reaction as described for Fpn-1 above, but without reducing agent. This resulted in a hinge region peptide (approx. 1096 Da) and Fc fragments without the hinge region peptide being obtained. To further isolate the hinge region peptide, Capture Select human IgG Fc was again used, acting to bind the Fc and leaving the 1096 Da peptide with FabRICATOR enzyme in the flow through fraction.

The peptide FabRICATOR fraction was analysed on a UHPLC system using a Zorbax RRHD 300SB-C18 reversed phase column at 215 nm detection. A synthetic 1096 Da hinge region peptide was used as a method control.

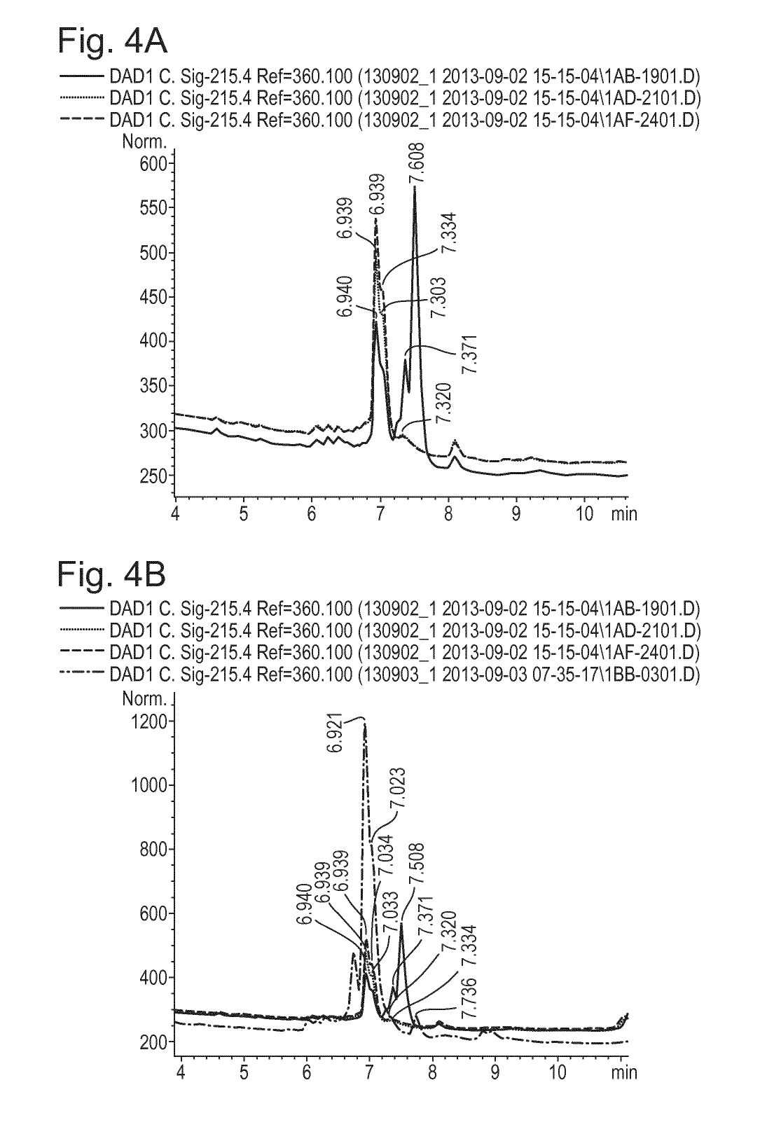

The chromatogram of FIG. 4A shows the 1096 Da peptide from preparations of adcetris, avastin and herceptin. The chromatogram of FIG. 4B also shows the synthetic peptide. Adcetris is an antibody drug conjugate with a conjugation site also in the hinge region. This preparation stands out with 2 additional peaks after reduction with TCEP, identified as conjugated variants of the hinge region.

Sequence listing
SEQ ID NO: 1
DQNFARNEKEAKDSAITFIQKSAAIKAGARSAEDIKLDKVNLGGELSGSN
MYVYNISTGGFVIVSGDKRSPEILGYSTSGSFDANGKENIASFMESYVEQ
IKENKKLDTTYAGTAEIKQPVVKSLLDSKGIHYNQGNPYNLLTPVIEKVK
PGEQSFVGQHAATGCVATATAQIMKYHNYPNKGLKDYTYTLSSNNPYFNH
PKNLFAAISTRQYNWNNILPTYSGRESNVQKMAISELMADVGISVDMDYG
PSSGSAGSSRVQRALKENFGYNQSVHQINRSDFSKQDWEAQIDKELSQNQ
PVYYQGVGKVGGHAFVIDGADGRNFYHVNWGWGGVSDGFFRLDALNPSAL
GTGGGAGGFNGYQSAVVGIKP
SEQ ID NO: 2
DSFSANQEIRYSEVTPYHVTSVWTKGVTPPANFTQGEDVFHAPYVANQGW
YDITKTFNGKDDLLCGAATAGNMLHWWFDQNKDQIKRYLEEHPEKQKINF
NGEQMFDVKEAIDTKNHQLDSKLFEYFKEKAFPYLSTKHLGVFPDHVIDM
FINGYRLSLTNHGPTPVKEGSKDPRGGIFDAVFTRGDQSKLLTSRHDFKE
KNLKEISDLIKKELTEGKALGLSHTYANVRINHVINLWGADFDSNGNLKA
IYVTDSDSNASIGMKKYFVGVNSAGKVAISAKEIKEDNIGAQVLGLFTLS
TGQDSWNQTN
SEQ ID NO: 3
CPPCPAPELLG
SEQ ID NO: 4
KTHTCPPCPAPELLGGPSVF
SEQ ID NO: 5
MADQNFARNEKEAKDSAITFIQKSAAIKAGARSAEDIKLDKVNLGGELSG
SNMYVYNISTGGFVIVSGDKRSPEILGYSTSGSFDANGKENIASFMESYV
EQIKENKKLDTTYAGTAEIKQPVVKSLLDSKGIHYNQGNPYNLLTPVIEK
VKPGEQSFVGQHAATGCVATATAQIMKYHNYPNKGLKDYTYTLSSNNPYF
NHPKNLFAAISTRQYNWNNILPTYSGRESNVQKMAISELMADVGISVDMD
YGPSSGSAGSSRVQRALKENFGYNQSVHQINRSDFSKQDWEAQIDKELSQ
NQPVYYQGVGKVGGHAFVIDGADGRNFYHVNWGWGGVSDGFFRLDALNPS
ALGTGGGAGGFNGYQSAVVGIKP
SEQ ID NO: 6
MRKRCYSTSAAVLAAVTLFVLSVDRGVIADSFSANQEIRYSEVTPYHVTS
VWTKGVTPPANFTQGEDVFHAPYVANQGWYDITKTFNGKDDLLCGAATAG
NMLHWWFDQNKDQIKRYLEEHPEKQKINFNGEQMFDVKEAIDTKNHQLDS
KLFEYFKEKAFPYLSTKHLGVFPDHVIDMFINGYRLSLTNHGPTPVKEGS
KDPRGGIFDAVFTRGDQSKLLTSRHDFKEKNLKEISDLIKKELTEGKALG
LSHTYANVRINHVINLWGADFDSNGNLKAIYVTDSDSNASIGMKKYFVGV
NSAGKVAISAKEIKEDNIGAQVLGLFTLSTGQDSWNQTN
SEQ ID NO: 7
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K

1. A method for analysing a sample of immunoglobulin molecules, comprising contacting the sample with a first polypeptide and a second polypeptide and analysing the resulting mixture, wherein the first polypeptide and the second polypeptide are cysteine protease enzymes which each cleave a different target site in the hinge region of human IgG.

2. A method according to claim 1, wherein the first polypeptide cleaves the hinge region of IgG between positions 238 and 239 according to the Kabat numbering system (positions 225 and 226 according to EU numbering system) and/or the second polypeptide cleaves the hinge region of IgG between positions 249 and 250 according to the Kabat numbering system (positions 236 and 237 according to EU numbering system).

3. A method according to claim 1, wherein the first polypeptide comprises or consists of the amino acid sequence of SEQ ID NO:1, or a variant or fragment thereof, and the second polypeptide comprises or consists of the amino acid sequence of SEQ ID NO: 2, or a variant or fragment thereof.

4. A method according to claim 1, wherein a variant of a said sequence is an amino acid sequence having at least 80% identity to said sequence, and a fragment of a said sequence comprises up to 300 contiguous amino acids of said sequence.

5. A method according to claim 1, wherein at least one immunoglobulin molecule in said sample is an IgG molecule, preferably a human IgG molecule.

6. A method according to claim 1, wherein at least one said IgG molecule is conjugated to a therapeutic agent, preferably via the thiol group of a cysteine residue of the IgG molecule.

7. A method according to claim 1, wherein said cysteine residue is in the hinge region of the IgG molecule.

8. A method according to claim 1, wherein the therapeutic agent is a cytotoxin, preferably selected from avristatin, a calicheamicin, CC-1065, doxorubicin, maytonsinoid, methotrexate and a vinca alkaloid.

9. A method according to claim 1, which comprises the steps:
(a) contacting said sample with said first polypeptide;
(b) isolating Fc fragments from the resulting mixture;
(c) contacting said isolated Fc fragments with said second polypeptide; and
(d) analysing the resulting mixture.

10. A method according to claim 1, which comprises the steps:
(a) contacting said sample with said second polypeptide;
(b) isolating Fab fragments from the resulting mixture;
(c) contacting said isolated Fab fragments with said first polypeptide; and
(d) analysing the resulting mixture.

11. A method according to claim 1, which comprises the steps:
(a) contacting said sample with both said first and said second polypeptide; and
(b) analysing the resulting mixture.

12. A method according to claim 1, wherein analysing the resulting mixture comprises determining the molecular weight of at least one molecule in the mixture, preferably via the use of high performance liquid chromatography (HPLC) and/or mass spectrometry.

13. A method according to claim 12, wherein said analysis comprises determining the presence, absence, and/or amount of a peptide with a molecular weight of approximately 1096 Da.

14. A method according to claim 13, wherein said peptide consists of the sequence CPPCPAPELLG (SEQ ID NO: 3), or a variant of said sequence comprising one or two conservative modifications, preferably wherein said modifications occur only within positions 2 to 10 of said sequence.

15. A method according to claim 1, wherein said analysis is carried out to determine:
(a) the proportion of immunoglobulin molecules in the sample to which a therapeutic agent is conjugated and/or unconjugated;
(b) the ratio of therapeutic agent:immunoglobulin molecule; and/or
(c) the presence or absence of post-translational modifications of the amino acid sequence set forth in SEQ ID NO: 3.

16. A peptide consisting of the amino acid sequence CPPCPAPELLG (SEQ ID NO: 3), or a variant of said sequence comprising one or two conservative modifications, preferably wherein said modifications occur only within positions 2 to 10 of said sequence; optionally wherein said peptide is conjugated to a therapeutic agent.

17. The peptide according to claim 16, which is produced by contacting an immunoglobulin containing sample with a first polypeptide and a second polypeptide as defined in claim 1.

18. A kit for use in a method of analysing a sample of immunoglobulin molecules, the kit comprising a first polypeptide and a second polypeptide as defined in claim 1.
